(19) United States
(12) Patent Application Publication
(10) Pub. No.: US 2022/0323879 A1
(43) Pub. Date: Oct. 13, 2022

(54) HUMUS TREATMENT PROCESS WITH ACTIVE NEUROLOGICAL SUBSTANCES

(71) Applicant: Suchad CHIARANUSSATI, Bangkok (TH)

(72) Inventor: Suchad CHIARANUSSATI, Bangkok (TH)

(21) Appl. No.: 17/765,811

(22) PCT Filed: Oct. 1, 2020
(86) PCT No.: PCT/TH2020/000068

§ 371 Date: Mar. 31, 2022

(30) Foreign Application Priority Data
Oct. 2, 2019 (TH) 1901006320

(51) Int. Cl. B01D 3/00; B01D 11/02; B01D 3/14; B01D 5/00; B09B 3/40

(52) U.S. Cl. CPC B01D 3/002; B01D 11/0288; B01D 11/0257; B01D 11/0296; B01D 3/143; B01D 5/0036; B01D 5/006; B09B 3/40

(57) ABSTRACT

According to aspect of the present invention there is provided humus treatment process with active neurological substances, that is, the waste treatment process in the pharmaceutical industry. That uses raw materials from plants. The process involves treatment of fossil plants, Canabis sative, Mittragyna speciose, Datura metel and Magic mushrooms in order to allow residues in the plant to interact with the system. Nerves are depleted or less than the set value and allowing the production waste to be used in the processing of the fibers and agriculture industry.

### TECHNICAL FIELD

The present invention relates to biotechnology in particular humus treatment process with active neurological substances.

### BACKGROUND

Plants are active in the nervous system can be categorized into 3 categories. 1. Sedatives and hypnotics, which help relieve anxiety, calm the nerves, and relieve seizures in some categories. 2. Psychopharmacologic agent acts on the central nervous system, reduces blood pressure, bradycardia, helps the work of the digestive system. But at the same time it will cause temporary mental and nervous disorders 3. Central nervous system stimulants have similar effects on human mental and behavioral conditions and it will not change mental conditions such as coffee or tea.

Canabis sative plants, such as Cannabis (Canabis sativa L. subsp. Indica or Indica), hemp (Canabis sativa L. subsp sativa or Sativa) etc. This plant is classified into group 1 that can find at south siberia, persia, china in the Himalayas and Kashmir. Then it spread to southeast asia and the tropical. Production of fiber from hemp in China. The Sativa taller than 2 meters. Indica is usually no more than 2 meters high. The essence of this plant is the Cana Boyd Boy (CBD) and Tetra Hyocanabinol (THC) which is commonly used in the treatment of diseases. That can find the patent regarding the substance of use, such as the request number PCT/GB2011/050487, PCT/DK2010/050341, PCT/GB2010/051066, etc. In determining the law on drug plants, Thailand will measure the THC value. That plants with THC less than 0.3% are not considered addicted, so hemp in the industry must have less than THC base.

Magic mushrooms or Psilocybin mushrooms were used in religious ceremonies. The celebrations popular in Europe and Africa found more than 180 species around the world. Mostly in Europe, Central America, India, south of Thailand, eat mushrooms, this group will have initial symptoms, intoxicating intoxication, unable to order direction when consumed from dried flowers, they are 50% less drunk than fresh flowers. The key substances are Psilocybin, Psilocin and Baeocystin, which are alkaloid-type sedatives (Alkaloidal indole), the same type of LSD (Acts LSD) makes the light visible in various colors as green and when the effect is done the color will true. Psilocybin and Psilozene is a substance used to control emotional expression and the feeling. If eater take too much, it will cause overjoyed and enchanted. These mushrooms are similar to the Galerinas or the deadly mushrooms. which are poisonous species of Cydopeptide. It destroys cells in the liver and kidneys. It cause liver failure and acute renal failure. Because the two groups of mushrooms are very similar in shape. Therefore it cause people to die from eating mushrooms. And due to difficulty in classifying. Therefore making mushroom selection difficult to extract and cure difficult diseases. So it must be planted in the control system and the mushroom is classified as Group 3 because it acts like a group that affects the mental state and human behavior but does not change the state of mind.

Kratom (Mittragyna speciose) has been found that the substance is Mitragynine, a substance in the Indole alkaloids. Therefore classified as group 2, resulting in numbness press fatigue, works longer and withstands more heat. If it exposed to the substance for a long time, the pigment will change and darken the skin. Kratom stalks and leaves are indigestible, that will settle in the intestine, it caused as a symptom. In ancient times, it was used as a drug to cure diarrhea, named Krasae Krathai in Thailand and used to treat opium addiction in New Zealand

Datura metel, group 2, is found in hyocyanin and hyocene, which acts as an anti-cholin ligament (Anticholinergic) in the central nervous system and the end nervous system, it causes dreamy symptoms, fast heartbeat, high blood pressure, fever, iris, enlarged mouth, dry throat. dry skin, decreased bowel movements And urinary retention. The treatment aside from sustaining danger from danger may also require the use of Physostigmine in patients with symptoms that may be fatal. And it may be used to help diagnose including the control of mania and dreaminess in some patients. This substance is found in all parts of the Datura metel. And it have narcotic effects. In the Thai traditional medicine, the use of the Datura metel as an ingredient in quitting narcotics produced from opium

The discovery of various chemicals in plants has led to the development of medicines and other chemical industries and creating virtual substances or mimicking derivatives which causes the quality of medicine. At the same time, the plants mentioned above are also popularly used as addict drugs. It cause human health problems, social problems, organized crime and national development. Then it must be careful protect and control the planting area. And solve the problem of substance abuse from plants. especially plant ones. For example, Datura metel, cannabis, marijuana, and mysterious mushrooms that have both active ingredients. Therefore must be disposed of by destroying but such destruction can also extract the active substance from waste in the destruction as well.

Patent number CN20140423, China, revealed the extraction process from marijuana by determining leaf and flower properties. Since it is a property determination of plants. it causes plants that do not meet the criteria and are left as waste and can only be used for cannabis not applicable to other plants that have active substances in the nervous system.

Patent number CN101121514, Zou Yan's China reveals the destruction of plant residues. Waste water treatment using oil separation method for extracting substances in plants which is a common type of crop destruction that does not use important substances from plant residues.

Patent Number. CN102048857, China, revealed how to extract alkaloids from Kratom by grinding Kratom leaves before extraction. But the process takes a long time.

Therefore, this invention will develop the extraction procedure to have a higher invention step. Which is applied to the destruction of humus that causes the whole trunk and shorten the separation time. It can also be used with plants that have many neurological active substances.

Currently, no patent is found, Nervous destruction of neurological plants. Especially plants that are beyond Cannabis, opium, such as Datura metel and magic mushrooms, etc.

### SUMMARY OF INVENTION

The purpose of this invention is to create a process for humus management that has concrete neurological active substances for use in the production of medical plants. And it prevent the leakage of narcotics through plant fossils that are used to produce drugs or to extract important substances Especially in different parts of plants. That do not use extracts but still have drugs spread even with a small amount. This will result in the expansion of the pharmaceutical industry that needs more active substances from the nervous system of plants. Without worrying about the release of substances from plant production sources. Especially the detachment by eliminating waste in the production process which causes social problems, consumer health and the distribution of drugs in society.

The technique and characteristics of the invention are the development of methods from extracting by developing a more advanced invention to suit the family of cananas, Datura metel and magic mushrooms.

### BRIEF DESCRIPTION OF THE DRAWINGS

FIG. 1 Diagram showing the process of humus management process with active neurological substances

### DETAILED DESCRIPTION

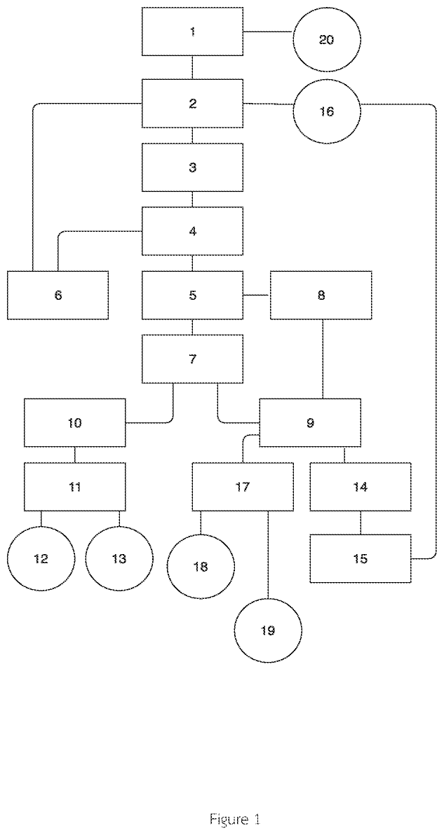

The Following Will Reveal the Process of Humus Management Process with Active Neurological Substances can be Explained as in FIG. 1 as Follows:

Procedure (1) take the remains of the crop or parts that have been harvested (20) to cut or chop or digest into fine pieces that are less than or equal to 5 millimeters in length. If it longer than 5 millimeters. the rounds of stirring in the next fermentation tank with a higher number of turns. It cause energy wastage after handling the humus from (20) until it having the above characteristics. That will call that plant material raw material, then take the raw material from step (1) into the stirring tank in step (2) and add solvent (16) to the stirring tank. and stir in step (3). It will rotate at an appropriate speed in the range of 1 to 200 rpm. If the total number is higher than 200 rpm, it will result in high energy consumption. Unsuitable for industrial systems the material is stirred, then stop stirring and leave for 10-20 minutes to ferment raw materials. The optimum duration is 15 minutes, which will cause the raw materials from the previous process to become fossils or discolored plant residues. It looks like a leaf that has been squeezed. The fermentation of this raw material is called step (4). When fermented in a specified time, the solvent liquids are mixed in the fermentation tank from the plant waste at step (5) and then separated. Solids and liquids separated. The liquid is stored in the tank (8) and the solids or waste are inspected for neurological substances that prevent leaks at step (7). The examination method depends on the type of substances of each plant by dividing into 2 ways which are:

Route 1, the above substances are more than or equal to 0.5 ppm, which is the residue go to step (6) which is mixed with the stirring tank in step (2) again.
Route 2, the substance is less than 0.5 ppm, which is to separate the solid and liquid mixed in again. In which the liquid is mixed with the liquid in the tank (8) to enter the process separation step (9) and the solid or the waste is removed from the fermentation tank into the waste destruction process (10).

The process of waste destruction in steps (10) enters one of the methods of drying by heating or cooling (11) depending on the type of plant or benefits to the processing of humus. In the event that the plant material cannot be processed, it will be packed for burning (12). In the case that the plant material can be processed, it will be packed for processing (13) next.

At step (9), the solution is separated by heating. The nervous system active substances of each plant will have different ranges of condensation points. It cause condensation distillation at different temperatures. Therefore. when heated, it results in separation of important substances and solvents. Vapor of those substances will separate along the pipe and condenses into solution in different areas. As for solvent vapor, it condenses at step (14) and combines into a solution at step (15). The solution obtained in that step is the solvent used in the process. And will be recycled combined with the step solvent (16) to be used in step (2).

At step (17), when the crude waste is obtained from the solution separation in step (9), the humus that is less than or equal to the value specified by the nervous system which depends on each type of plant. There are two methods of destruction that can be chosen from packaging for destruction by the use of chemicals in steps (18) or for packaging for processing purposes (19).

The method for the separation of the solution by heating the steps (9) is not appropriate to use conventional distillation methods. Because a one-time distillation makes the important substances that act on the nervous system which not leaving much plant matter. It due to the various substances in each plant and there is distillation at different temperatures, so when one distillation is set and determine the temperature at once boiling point. That will cause some substances to condense at different temperatures. In addition, the above method also applies to mixtures where each substance in the mixture has a boiling point of 80 degrees Celsius or more. Which is an important substance that affects the nervous system in some plants. It has a similar boiling point and less than 80 degrees Celsius. Therefore, the separation of the solution in steps (9) aside from being not suitable for normal distillation.

The method of heating the solution in separation (9) by fractional distillation is inappropriate. Due to the boiling point of the substance to be separated. There is not much difference in temperature. Therefore, the temperature of boiling point must be determined precisely to get the pure substance. And because there are many important substances. the set temperature are many numbers. It depending on the amount of substance to be separated. Which according to the definition of distillation, it will be used to separate mixtures of two or more substances mixed together. Therefore, the boiling point temperature specified must have more than 2 numbers as well, i.e. at least 3 numbers, in order to obtain the important substance on each floor of the distillation tower, have enough purity substance which is different from this invention. Because although there are many kinds of substances in plants that we want to separate from humus. But we are mainly interested in the separation of neurotransmitters. Therefore. the constant temperature must be determined precisely, not the number group, such as the boiling point temperature in the distillation sequence. But the only numeric value is the highest or lowest value of the fixed temperature group. Therefore, the temperature value of the numbers is only 1 or 2. The numbers are then compared to the boiling point of the solvent to find the desired value. It depending on the plant. as will be the case in the next order, so the method of this invention is clearly different from fractional distillation.

Another important reason there are many important substances in plants which has not much difference in boiling point. But at the same time, there will be some plants that have neurotransmitters active at different temperatures. And such methods are commonly used in the petroleum industry to refine crude oil which is different from the separation of important substances from plant residues. Because in the agricultural industry do not need to make a large distillery and use a furnace with temperatures up to 300 degrees Celsius or more.

Therefore, it can be concluded that both normal distillation method and segment distillation method. It is not suitable enough to be used as a step (9). The method of separation of the solution by heating the solution separation step (9).

Heating to the evaporation point of the material to be separated cause the vapor to float into the distillation equipment which has a high style. The solution vapor will float from the top of the distillation device. As mentioned, the rooms are divided horizontally with a concave partition. And it has a hole to allow hot vapor solution can pass to the next room by passing through the top to the bottom of the distillation equipment. Each room has a solution with different condensation points which will separate the pipe and condense at the step (14).

In the case of Cannabis, the solvent (16) such as isopropyl alcohol (C3H8O), methanol (CH4O), acetone (C3H6O), Ethanol (C2H6O), Heptane (Hethene or C7H16) The number of rooms in the distillation apparatus of the appropriate procedure (9) is 2-11 rooms to divide the solution with different condensation points. Because there are more than 120 active substances in marijuana and hemp, and some neurotransmitters. The number of rooms in the distillation apparatus of the procedure (9) should not be less than 2 rooms for the separation of THC out of other substances. Because THC is an important neurotransmitter. And it has been specified in drug laws throughout Thailand. The number of rooms in the distillation apparatus of the procedure (9) should not be greater than 11, because the active substance is related to the nervous system in Cannabis and hemp. According to the study and research, there are about 10 types. The most suitable room is 6 rooms to separate substances, such as THC (Dealta-9 Tetrahydrocannabinol or THC) Cannabidol (CBD), and Canabico. Cannabichromene (CBC), CBN (CBN or C21H26O2), Terpenes and Flavonoids, which are demanding in the pharmaceutical industry to leave other substances. Because the residual substances in the humus will be able to bring the humus to extract later. Which increases the chance of escaping the release of important neurotransmitters. The optimum temperature for heating (9) steps is in the range of 50 to 260 degrees Celsius, so that the solvent with the lowest boiling point is acetone. Also, the evaporation point of the neurotransmitters is more than 50 degrees Celsius. And the temperature should not exceed 260 degrees Celsius because it is the lowest temperature. That causes flavonoid substances to evaporate. Although this group is not a neurotransmitter, it is an important substance in the cosmetic industry and some substances have evaporation points similar to those in the nervous system. Another reason, if temperature is higher than 260 degrees Celsius, it will cause the hums to not be able to be used in the fiber industry. The fibers from hemp are better quality than cotton high toughness and flexibility. Therefore, the selection of suitable plant disposal processes will be divided into
1. Once the crude is obtained from step (17), select the step (19)
2. After step (11), select step (13) that will be selected to use the humus in the fiber industry. But in the absence of processing, the procedure (18) should be used

In the case of kratom (Mittragyna speciose), suitable solvents (16) include alcohol. chloroform (CHCl3) and acetic acid (C3H4O2).

The number of rooms in the distillation apparatus of the procedure (9) for the cottage. The optimum is 2-9 rooms to divide the solution with different condensation points. If less than 2 rooms, it will not be able to separate any substances. If more than 9 rooms, it will make it unsuitable because too many substances to be separated. The optimum number of rooms is 3 rooms for separation of substances, including Mitragynine, Speciogynine, Paynantheine, Specoceleine, Flavonoid, Terpenoid, Saponin and 7-Hidroxymitragynine. Although there are 7 important substances, but because many substances have the same evaporation point. they can be divided into 3 chambers. The optimum temperature for heating the process (9) is in the range of 60 to 260 degrees Celsius in order to Causing the solvent with the lowest boiling point to be chloroform (CHCl3) evaporated completely. And the temperature should not exceed 260 degrees Celsius because it is sufficient temperature causing flavonoid substances to evaporate. Humus from Kratom is not suitable to use destroying by chemicals. Because Kratom can also be used to make planting materials for the agro-industry such as making artificial soil. The material of the orchid island, etc. therefore should not use chemicals. But it should be processed back for further use. Therefore, the selection of suitable plant disposal processes will be divided into
1. Once the crude is obtained from step (17) select the step (19)
2. When passing through step (11), choosing step (13) to use vegetable fossil for agricultural industry

In the case of magic mushrooms, suitable solvents (Methanol or CH4O), water, ethanol (C2H6O), the most suitable solvent (16) is available Methanol (CH4O). The number of rooms in the distillation apparatus of the step (9) for the appropriate magic mushroom is 2-5 rooms to divide the solution with different condensation points. If less than 2 rooms, it will not able to separate any substances. The most suitable room is 5 rooms to separate substances, namely Psilocybin. Psilocin and Baeocystin, which is a neurotransmitter. Because of the suitable solvent property, it give results of clear separation of different substances. In particular, the use of ethanol (Ethanol or C2H6O) to make the separation of Psilocybin from the mushroom remains little. Therefore, it have to add solvents such as water to be used together. As a result, the optimal number of rooms is 5 rooms. The optimum temperature for heating (9) steps is in the range of 60 to 260 degrees Celsius, so that the solvent with the lowest boiling point is ethanol Mol (Methanol or CH4O) completely evaporated. And the temperature should not exceed 260 degrees Celsius. Because the evaporation point of Baeocystin when using a solvent with methanol (Methanol or CH4O) will reach 254 to 258 degrees Celsius. Therefore, if the temperature is higher than this, it will use a waste of energy resources. Due to the small amount of the remains of the astonishing mushrooms, the fibers cannot be used in industry. In addition, astonishing mushrooms are plants that are used for consumption. Therefore, the selection of suitable plant disposal processes will be divided into
1. When obtaining a crude (Crude) from step (17) then choose step (18) or choose to burn
2. When step (11) is selected, step (12)

Datura metel, the suitable solvents (16) is water. ethanol (C2H6O), ether, petroleum ether, alcohol, chloroform. (Chloroform or CHCl3), benzene. The most suitable solvent (16) is water because it is conveniently supplied and dissolves all important substances well. The number of rooms in the distillation equipment of the procedure (9) for Datura metel is appropriate for 2-4 rooms. The most suitable room is 4 to separate the Hyoscyamine and hyoscine. The optimum temperature for heating (9) steps is in the range of 30 to 120 degrees Celsius, so that the solvent with the lowest boiling point is ether evaporate completely. And the temperature should not exceed 120 degrees Celsius, so that the two important substances are separated. Because Datura metel is a tree that contains neurotransmitters active throughout the trunk. Therefore must take all parts of the trunk into the humus management process. To prevent the release of active substances on the nervous system, the remains of the Datura metel can be used to make planting materials. Therefore, the selection of suitable plant disposal processes will be divided into
1. Once the crude is obtained from step (17) choose the step (19)
2. When passing through step (11), choosing step (13) to use vegetable fossil for agricultural industry

1. Humus treatment process with active neurological substances comprising;
steps (1) take the remains of the crop or parts that have been harvested (20) into sliced or chopped or sub-sliced into fine pieces that are less than or equal to 5 millimeters in length then handling the carcass from (20) until it looks As mentioned, it will call that plant material raw material, then take the raw material from step (1) into the stirring tank in step (2) and add solvent (16) to the stirring tank, will stir in step (3) and it will rotate at an appropriate speed in the range of 1 to 200 rpm when the material is stirred and it will stop stirring and leave for 10-20 minutes to ferment raw materials; the fermentation of this raw material is called step (4) then the solvent liquid is separated from the waste in step (5) and then separated from solids together, the liquid will be stored in the tank (8) and the solids or residue will be examined for neurological substances that prevent leaks at step (7) by separating into 2 ways which are:
route 1, the above substances are more than or equal to 0.5 ppm, which is the residue go to step (6) which is mixed with the stirring tank in step (2) again;
route 2, the substance is less than 0.5 ppm, which is to separate the solid and liquid mixed in again. In which the liquid is mixed with the liquid in the tank (8) to enter the process separation step (9) and the solid or the waste is removed from the fermentation tank into the waste destruction process (10). The process of destroying the waste in steps (10) enters the thermal or cold drying method (11);
if humus cannot be processed, it will be packed and burned. (12) If the humus is processed, it will be packing for processing (13);
at step (9), the solution is separated by heating, the neurological active substances of plants are the range of condensation points is different. It cause condensation distillation at different temperatures. Therefore, when heated. it results in separation of important substances and solvents. Vapor of those substances separate along the pipe. And condenses into solution in different areas. As for solvent vapor, it condenses at step (14) and combines into a solution at step (15). The solution obtained in that step is the solvent used in the process. And it will be recycled combined with the step solvent (16) to be used in step (2);
at step (17), when the crude waste is obtained from the solution separation in step (9), the humus that is less than or equal to the value specified by the nervous system, there are two methods of destruction that can be chosen from packaging for destruction by the use of chemicals in steps (18) or for packaging for processing purposes (19);
the characterized by heating in the solution separation process in the step (9) will heat up to the evaporation point of the material to be separated, it cause the vapor to float into the distillation equipment, the solution vapor will float from the top of the distillation device in internal distillation equipment;
as mentioned, the rooms are divided horizontally with a concave partition, and has a hole to allow hot vapor solution can pass to the next room by passing through the top to the bottom of the distillation equipment, each room has a solution with different condensation points, which will separate the pipe and condense at the step (14).

2. Humus treatment process with active neurological substances in accordance with claim 1 wherein the solvent (16) choose from isopropyl alcohol, Isopropyl alcohol or C3H8O, Methanol or CH4O, Acetone or C3H6O, Ethanol or C2H6O), Hethene (C7H16), Chloroform (CHCl3), Acetic Acid (C3H4O2) or water.

3. Humus treatment process with active neurological substances in accordance with claim 1 wherein the number of rooms in the distillation equipment of the suitable procedure (9) is 2-11 rooms for the canabis plants and the optimum temperature for heating the process (9) is between 50 and 260 Degrees Celsius, which has the selection of plant disposal methods as follows:
route 1, when obtaining a crude from step (17), then choose step (19);
route 2, after passing step (11) choose step (13) but in the case of no processing choose step (18).

4. Humus treatment process with active neurological substances in accordance with claim 1, wherein the number of rooms in the distillation equipment of the procedure (9) for Kratom, the optimum is 2-9 rooms For Kratom (Mittragyna speciose) and the optimum temperature for heating in (9) steps is in the range of 60 to 260 degrees Celsius. the disposal procedures for the crop are as follows:
route 1, when getting crude from step (17), choose step (19);
route 2, after step (11), choose step (13).

5. Humus treatment process with active neurological substances in accordance with claim 1 wherein, the number of rooms in the equipment for the extraction of the steps (9) is suitable for 2-5 rooms for the magic mushroom and the optimum temperature for heating the steps (9) is in the range of 60 to 260) Celsius Degree, which has the selection of the process of disposal of plants as follows;
route 1, when the crude waste from step (17), then choose the step (18) or choose to burn;
route 2, go through the steps (11) that choose the step (12).

6. Humus treatment process with active neurological substances in accordance with claim 1 wherein, the number of rooms in the distillation equipment of step (9) is suitable for 2-4 rooms for Datura metel and the optimum temperature for heating in step (9) is in the range of 30 to 120 Degrees Celsius, which has the selection of plant disposal methods as follows:
route 1, when obtaining a crude from step (17), then choose step (19);
route 2, after passing step (11) choose the procedure (13).
